(19) United States
(12) Patent Application Publication
(10) Pub. No.: US 2015/0240239 A1
(43) Pub. Date: Aug. 27, 2015

(54) METHODS AND PHARMACEUTICAL COMPOSITIONS FOR INHIBITING MATRIX METALLOPROTEINASES 1 BY INTERFERENCE RIBONUCLEIC ACID

(71) Applicant: Nugen Biosience (Taiwan) Co., Ltd., Taipei (TW)

(72) Inventors: Shann-Tzong Jiang, Taipei (TW); Gen-Hung Chen, Taichung (TW); Chun-Hua Huang, Kaohsiung (TW)

(73) Assignee: Nugen Biosience (Taiwan) Co., Ltd., Taipei (TW)

(21) Appl. No.: 14/191,628

(22) Filed: Feb. 27, 2014

(51) Int. Cl. C12N 15/113

(52) U.S. Cl. CPC C12N 15/1137

(57) ABSTRACT

An interference ribonucleic acid containing specific sequence for inhibiting MMP1, which is composed of 19 to 25 nucleotides, wherein the interference ribonucleic acid has a sequence selected from the group consisted of the sequences as set forth in, or essentially identical to, SEQ ID NO: 5, SEQ ID NO: 6, SEQ ID NO: 7, SEQ ID NO: 8, SEQ ID NO: 9, and SEQ ID NO: 10. By means of the interference ribonucleic acid and MMP1 with high specificity binding, gene and protein expression of endogenous MMP1 of cancer cell can be inhibited, and then the interference ribonucleic acid can be used for pharmaceutical composition to prevent or treat cancer metastasis or to improve wrinkle resistance.

### BACKGROUND OF THE INVENTION

1. Field of the Invention

The present invention relates to an interference ribonucleic acid, and more particularly relates to an interference ribonucleic acid for inhibiting MMP1 expression.

2. Description of the Prior Arts

Matrix metalloproteinases (MMPs) are zinc-dependent endopeptidase. MMPs are involved in the degradation of extracellular matrix, and are associated with increased skin wrinkles, reduced wound healing and enhanced tumor metastasis. In normal physiological processes, the activity of MMPs would be affected by pro-proenzyme activation, endogenous inhibitors, α-macroglobulins, and endogenous matrix metalloproteinase inhibitor (MMPI). MMPs family has very similar characteristic, and an archetypal structure from N-terminal consisting of a signal peptide domain (about 80 amino acids), a pro-peptide domain, a catalytic domain (about 170 amino acids), a linker region and a hemopexin-like domain to C-terminal.

Matrix metalloproteinases-1 (MMP-1) of MMPs family is a zinc-dependent endopeptidase, wherein a sulfhydryl group of cysteine residue (conserved sequence, PRCGVPD) of propeptide domain would bind to zinc ion of catalytic domain, thus inhibiting MMP-1 and maintaining an inactive state (proenzyme). While cysteine residue is replaced or treated by chemical agent to break Cys-Zn²⁺ binding, MMP-1 would be activated. MMP-1 is one of the tryptases to degrade mainly extracellular matrix from dermis, specifically, the types III collagens. Moreover, MMP-1 also can degrade casein and gelatin.

Cancer, medically known as a malignant neoplasm, is a broad group of diseases involving a series of cell unregulated division or growth, and invasion to form malignant neoplasm. The characteristics of cancer cell have been proposed: sustaining proliferative signaling, evading growth suppressors, resisting cell death, enabling replicative immortality, inducing angiogenesis, and activating invasion and metastasis. The progression of metastasis of cancer cell involves multiple forms as follows: (1) cancer cell would overcome attachment with endothelial cell to detach itself from metrocyte; (2) partial cancer cell would undergo anoikis due to apoptosis; (3) cancer cell would degrade protein of extracellular matrix (ECM) to penetrate into basement membrane; (4) cancer cell would penetrate into barrier under basement membrane and move within extracellular matrix; (5) cancer cell would penetrate into lymph nodes and blood vessels to invade cell; (6) cancer cell would penetrate into the walls of blood vessels to circulate through the bloodstream and avoid immune cell from tracking; (7) cancer cell would attach to endothelial cells; (8) cancer cell would extravasate from lymph nodes and blood vessels; (9) cancer cell would proliferate in target tissue and induce angiogenesis to supply nutrient and oxygen for cancer cell, and then destroy normal cell to cause host death.

MMP-1 plays an important role of metastasis and skin aging with regard to MMP-1 preliminary degrading collagen of extracellular matrix, and is an interstitial collagenase.

### SUMMARY OF THE INVENTION

To overcome the shortcomings of the prior art such as MMPs inhibition in vain result in skin shrinkage and aging and cancer cell proliferation, the objective of the present invention is to provide an interference ribonucleic acid for inhibiting MMP1, more particularly to provide a specific sequence interference ribonucleic acid (RNAi) to inhibit MMP1 expression, and then inhibit cancer cell deterioration and metastasis.

Therefore, in one aspect, the present invention provides an interference ribonucleic acid for inhibiting MMP1, which is composed of 19 to 25 nucleotides, and the interference ribonucleic acid has a sequence essentially identical to the sequence as set forth in SEQ ID NO: 5 or the sequence as set forth in SEQ ID NO: 6.

According to the present invention, the term “specific sequence”, as used herein, refers to at least 19 to 25 consecutive nucleic acids of the sequence between positions +144 and +1553 set forth in MMP1 messenger RNA (mRNA); in one embodiment, the sequence of mRNA is NCBI Reference Sequence NM 002421.3. While the sequence between positions +144 and +1553 of the mRNA sequence translates into a protein, it presents the catalytic domain of MMP1 protein.

The term “essentially identical”, as used herein, refers to degree of similarity between two sequences, which is determined by identical and/or conservative ratio between the sequences. It will be understood that the variation of nucleic acid or amino acid sequence does not necessarily affect the genetic code. Thus, one of skill in the art will recognize that a nucleic acid or an amino acid encoded by said target gene having certain homology from its unperturbed level is included in the present invention.

Preferably, the degree of homology between the interference ribonucleic acid and the sequence as set forth in SEQ ID NO: 5 or the sequence as set forth in SEQ ID NO: 6 is at least 90%. More preferably, the degree of homology between the interference ribonucleic acid and the sequence as set forth in SEQ ID NO: 5 or the sequence as set forth in SEQ ID NO: 6 is at least 98%.

The present invention further provides an interference ribonucleic acid for inhibiting MMP1, which is composed of 19 to 25 nucleotides, and the interference ribonucleic acid has a sequence essentially identical to the sequence as set forth in SEQ ID NO: 7 or the sequence as set forth in SEQ ID NO: 8.

Preferably, the degree of homology between the interference ribonucleic acid and the sequence as set forth in SEQ ID NO: 7 or the sequence as set forth in SEQ ID NO: 8 is at least 90%. More preferably, the degree of homology between the interference ribonucleic acid and the sequence as set forth in SEQ ID NO: 7 or the sequence as set forth in SEQ ID NO: 8 is at least 98%.

The present invention also provides an interference ribonucleic acid for inhibiting MMP1, which is composed of 19 to 25 nucleotides, and the interference ribonucleic acid has a sequence essentially identical to the sequence as set forth in SEQ ID NO: 9 or the sequence as set forth in SEQ ID NO: 10.

Preferably, the degree of homology between the interference ribonucleic acid and the sequence as set forth in SEQ ID NO: 9 or the sequence as set forth in SEQ ID NO: 10 is at least 90%. More preferably, the degree of homology between the interference ribonucleic acid and the sequence as set forth in SEQ ID NO: 9 or the sequence as set forth in SEQ ID NO: 10 is at least 98%.

The present invention further provides a method of inhibiting MMP1 in a subject in need thereof, comprising: administering an effective amount of an interference ribonucleic acid having a length of 19 to 25 nucleotides to the subject, wherein the interference ribonucleic acid has a sequence selected from the group consisting of the sequences as set forth in, or essentially identical to, SEQ ID NO: 5, SEQ ID NO: 6, SEQ ID NO: 7, SEQ ID NO: 8, SEQ ID NO: 9, and SEQ ID NO: 10.

The present invention further provides a pharmaceutical composition for anti-cancer metastasis comprising an effective amount of an interference ribonucleic acid, and a pharmaceutically acceptable carrier, wherein the interference ribonucleic acid has a sequence selected from the group consisted of the sequence as set forth in, or essentially identical to, SEQ ID NO: 5, SEQ ID NO: 6, SEQ ID NO: 7, SEQ ID NO: 8, SEQ ID NO: 9, and SEQ ID NO: 10.

According to the present invention, the term “anti-cancer metastasis”, as used herein, refers to inhibiting or relieving cancer cell from a primary site to a second site. The term “effective amount” as used herein, refers to a dosage to alleviate or inhibit progress of cancer. According to the present invention, the effective amount for inhibiting protein expression of endogenous MMP1 of cancer cell is determined by administering the interference ribonucleic acid in a specific amount and time to inhibit MMP1 gene and its protein expression.

Preferably, the pharmaceutically acceptable carriers include water, buffer, salt, amino acid, glycerol, hyaluronic acid or mannitol. In accordance with the present invention, a pharmaceutical composition for anti-cancer metastasis, as a transfection reagent for introducing RNAi into a host by transfection is prepared, and then administrated to a subject by injection, such as intravenous, intrathecal, periganglionic, intraventrical, intraparenchymal, intramuscular injection or subcutaneous injection.

Preferably, the effective amount of the interference ribonucleic acid is administered at a dose of 0.5 μg to 1.5 μg.

Preferably, the cancer includes, but is not limited to, lung cancer, prostate cancer, breast cancer, colon cancer, gastric cancer, pancreatic cancer, liver cancer, esophageal cancer, brain tumor, ovarian cancer, uterine cancer, melanoma, kidney cancer, head and neck tumor, skin cancer, bladder cancer, osteosarcoma, biliary tract cancer, vulvar cancer, testicle tumor, penis cancer, rectal cancer, mediastinal tumor, urethra cancer, villous cancer, thyroid cancer, parathyroid carcinoma, pheochromocytoma, blastocytoma, malignant lymphoma, leukemia and multiple myeloma.

Preferably, the skin cancer is basal cell carcinoma (BCC), squamous cell carcinomas (SCC) or melanoma.

In one embodiment, RNA interference (RNAi) is a process by which double-stranded RNA (dsRNA) is used to silence gene expression. The steps comprise the following: RNAi begins with the cleavage of longer dsRNAs into small interfering RNAs (siRNAs) by an RNaseIII-like enzyme, dicer. SiRNAs are ssRNAs that are usually about 19 to 25 consecutive nucleotides in length. SiRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs and specifically regulates gene expression to cause post-transcriptional gene silencing (PTGS).

Since siRNA can knock down specific gene to cause gene silencing by various transfections, siRNA binding to a target mRNA sequence is used as an important tool for gene functional study and target medicine.

Said transfection includes, but are not limited to, liposomal transfection, chemical transfection, DNA ligation, transgene insertion, chemical transformation, electroporation, homologous recombination, transposon insertion, jumping gene transfection, retrovirus infections, micro-injection, gene-gun penetration, and combinations thereof.

The length of the interference ribonucleic acid of the present invention is 19 to 25 nucleotides, and the content of guanine and cytosine (GC) is about 30% to 50%. By means of the interference ribonucleic acid and MMP1 binding with high specificity after transfection, gene and protein expression of endogenous MMP1 of cancer cell can be inhibited, and then the interference ribonucleic acid can be used for preventing or treating cancer metastasis or improving wrinkle resistance.

### BRIEF DESCRIPTION OF THE DRAWINGS

FIG. 1 provides representative images showing phenotype and fluorescence of MeWo cells transfected with 357MMP1-pAcGFP1-N3 vector for 24 hours, 48 hours, and 72 hours; wherein the images from top to bottom in the left column are bright field images respectively for 24 hours, 48 hours, and 72 hours; wherein the images from top to bottom in the right column are fluorescence images respectively for 24 hours, 48 hours, and 72 hours;

FIG. 2 is the result of quantitative analysis of MeWo cells transfected with 357MMP1-pAcGFP1-N3 vector of various concentrations (0.5 μg, 0.75 μg, 1.0 μg, and 1.5 μg) for 24 hours, 48 hours, and 72 hours;

FIG. 3 provides fluorescence images of MeWo cells co-transfected with 357MMP1-pAcGFP1-N3 vector (column A), 710MMP1-pAcGFP1-N3 vector (column B) or 742MMP1-pAcGFP1-N3 vector (column C) and control siRNA of various concentrations (10 pmole, 30 pmole, 50 pmole, 70 pmole, and 90 pmole) for 48 hours;

FIG. 4A illustrates the fluorescent intensity and cell survival rate of MeWo cells co-transfected with 357MMP1-pAcGFP1-N3 vector and control siRNA of various concentrations (10 pmole, 30 pmole, 50 pmole, 70 pmole, and 90 pmole) for 48 hours;

FIG. 4B illustrates the fluorescent intensity and cell survival rate of MeWo cells co-transfected with 710MMP1-pAcGFP1-N3 vector and control siRNA of various concentrations (10 pmole, 30 pmole, 50 pmole, 70 pmole, and 90 pmole) for 48 hours;

FIG. 4C illustrates the fluorescent intensity and cell survival rate of MeWo cells co-transfected with 742MMP1-pAcGFP1-N3 vector and control siRNA of various concentrations (10 pmole, 30 pmole, 50 pmole, 70 pmole or 90 pmole) for 48 hours;

FIG. 5 provides fluorescence images of MeWo cells co-transfected with 357MMP1-pAcGFP1-N3 vector (column A), 710MMP1-pAcGFP1-N3 vector (column B) or 742MMP1-pAcGFP1-N3 vector (column C) and target siRNA of various concentrations (10 pmole, 30 pmole, 50 pmole, 70 pmole or 90 pmole) for 48 hours;

FIG. 6A illustrates the fluorescent intensity and cell survival rate of MeWo cells co-transfected with 357MMP1-pAcGFP1-N3 vector and target siRNA of various concentrations (10 pmole, 30 pmole, 50 pmole, 70 pmole or 90 pmole) for 48 hours;

FIG. 6B illustrates the fluorescent intensity and cell survival rate of MeWo cells co-transfected with 710MMP1-pAcGFP1-N3 vector and target siRNA of various concentrations (10 pmole, 30 pmole, 50 pmole, 70 pmole or 90 pmole) for 48 hours;

FIG. 6C illustrates the fluorescent intensity and cell survival rate of MeWo cells co-transfected with 742MMP1-pAcGFP1-N3 vector and target siRNA of various concentrations (10 pmole, 30 pmole, 50 pmole, 70 pmole or 90 pmole) for 48 hours;

FIG. 7A illustrates the electrophoresis of MeWo cells transfected with 357siRNA or 710siRNA of various concentrations (100 pmole, 300 pmole, 500 pmole, 700 pmole or 900 pmole) by real-time quantitative polymerase chain reaction (RT-PCR); wherein glyceraldehyde 3-phosphate dehydrogenase (GADPH) is used as internal control;

FIG. 7B is the bar chart of FIG. 7A after normalization; and

FIG. 8 illustrates the electrophoresis of MeWo cells transfected with 357siRNA or 710siRNA of various concentrations (10 pmole, 30 pmole, 50 pmole, 70 pmole or 90 pmole) by Western blot analysis; wherein GADPH is used as internal control.

### DETAILED DESCRIPTION OF THE PREFERRED EMBODIMENTS

Other objectives, advantages and novel features of the invention will become more apparent from the following detailed description when taken in conjunction with the accompanying drawings.

The objective of the embodiments is to construct transgenic vectors suitable to bind target gene specifically by genetic engineering, to analyze the efficacy of gene silencing by co-transfecting each transgenic vector into mammalia cell, to select siRNA having gene silencing, and to observe the inhibition effect of siRNA relative to endogenous MMP1 genes by quantitative real time polymerase chain reaction (qPCR) and western blotting.

According to the present invention, the term “polymerase chain reaction (PCR)”, as used herein, refers to that a mixture comprising primers, template, polymerase, deoxyribonucleotide triphosphate (dNTP) and reaction buffer goes through an amplification to obtain an amplification product (PCR product).

According to the present invention, the term “vector”, as used herein, refers to any recombinant vector, is a recombinant expression system, and can express a specific nucleic acid in any host system constitutively or inducibly. The recombinant expression system may or may not possess self-replicating, and may be merely capable of transient expression in host cell.

According to the present invention, the term “primers”, as used herein, refers to preparation by any known chemical or biological synthesis process in the art. For example, while the primers are made of nucleic acid, they can be prepared by common nucleic acid synthesis such as deoxyribonucleic acid (DNA) synthesizer.

According to the present invention, the term “complementary” or “complementation”, as used herein, refers to a complementary nucleotide acid. For example, sequence “ACT” is complementary to sequence “TCA” or “TCU”. Complementation can be between two strands DNAs, between DNA and RNA, or between two strands RNAs. Base pairs of nucleotide acid can pair partially or completely. The complementary level of pairing will significantly affect hybridization efficiency.

### Preparation Example 1

### Extraction of Complementary DNA (cDNA) from D551 Cell

D551 (human fibroblasts) cells obtained from Food Industry Research and Development Institute (Taiwan) (BCRC 60118) were removed from α-MEM medium, and then UltraspecII RNA™ isolation kit was utilized to extract to obtain ribonucleic acid (RNA). 2 μl RNA, 5 μl (25 mM) oligo dT (12-18 base), and 1 μl diethyl pyrocarbonate water (DEPC water) were mixed and spanned down to form a mixture. The mixture was heated at 70° C. for 5 minutes, and cooled at 4° C. to obtain a product. 4 μl product, 5.0 μl 5× volume reaction buffer, 1.0 μl (25 mM) dNTPs, 4.0 μl (25 mM) MgCl₂, 1.0 μl RNase OUT (20˜40 Unit/μl), 2.5 μl AMV reverse transcriptase, and 7.5 μl DEPC water were mixed at 25° C. for 5 minutes, 42° C. for 60 minutes, 72° C. for 10 minutes, and ended at 4° C. for cooling to obtain a first strand cDNA.

### Preparation Example 2

### Construction of Target Gene and Plasmid

(1) MMP1-pGEM Easy Vector

The first strand cDNA confirmed by agarose gel electrophoresis and purification was used as a template. The template, forward primer 5′-ATGCACAGCTTTCCTCCACTGCT-3′ (SEQ ID NO. 1) and reverse primer 5′-TCAATTTTTCCTGCAGTTGAACCAGCTAT-3′ (SEQ ID NO. 2) were mixed to go through PCR and combined with pGEM-T Easy vector to form a transgenic vector, and presented as a MMP1-pGEM Easy vector.

(2) MMP1-pAcGFP1-N3 Vector

The MMP1-pGEM Easy vector was used as a template. The template, forward primer 5′-AAGCTTGCCGCCACCATGGGTAGCTTTCCTCCACTGCTGCTG-3′ (SEQ ID NO. 3) (HindIII site was marked in italics) and reverse primer 5′-GGATCCGATGGGCTGGACAGGATTTTGGGAACGTCCATATATGGC-3′ (SEQ ID NO. 2) (BamHI site was marked in italics) were mixed to go through PCR to obtain a PCR product. The PCR product was ligated to pAcGFP1-N3 cut by HindIII and BamHI to obtain a MMP1-pAcGFP1-N3 vector (831 bp), and then MMP1-pAcGFP1-N3 vector was cut by HindIII and BamHI to obtain a pAcGFP1-N3 vector having two cut sites.

(3) 357MMP1-siRNA-pAcGFP1-N3 Vector

The primers as shown in Table 1 were modified to construct 357MMP1-siRNA-pAcGFP1-N3 vector, 710MMP1-siRNA-pAcGFP1-N3 vector, and 742MMP1-siRNA-pAcGFP1-N3 vector separately.

TABLE 1
The primer pairs of 357MMP1-siRNA-pAcGFP1-N3 vector,
710MMP1-siRNA-pAcGFP1-N3 vector, and 742MMP1-siRNA-
pAcGFP1-N3
vector

| Vector | | primers |
|---|---|---|
| 357MMP1-siRNA-pAcGFP1-N3 | SEQ ID NO. 5 | CACGCCAGATTTGCCAAGAGCAGATC |
| | SEQ ID NO. 6 | GATCTGCTCTTGGCAAATCTGGCGTG |
| 710MMP1-siRNA-pAcGFP1-N3 | SEQ ID NO. 7 | GCTACACCTTCAGTGGTGATGTTCA |
| | SEQ ID NO. 8 | TGAACATCACCACTGAAGGTGTAGC |
| 742MMP1-siRNA-pAcGFP1-N3 | SEQ ID NO. 9 | CAGGATGACATTGATGGCATCCAAGG |
| | SEQ ID NO. 10 | CCTTGGATGCCATCAATGTCATCCTG |

5′ end of SEQ ID NO. 5 in Table 1 was dephosphorylated and then bound to HindIII cut site (in italics) to form a forward primer (5′-AGCTCACGCCAGATTTGCCAAGAGCAGATC-3′); 5′ end of SEQ ID NO. 6 in Table 1 was also dephosphorylated and then bound to BamHI cut site (in italics) to form a reverse primer (5′-GATCGATCTGCTCTTGGCAAATCTGGCGTG-3′). 5 μl of the forward primer and 5 μl of the reverse primer were annealing at 75° C. in PCR to form a double strand. 4 μl double strand and 2 μl pAcGFP1-N3 were ligated at 4° C. to obtain a 357MMP1-siRNA-pAcGFP1-N3 vector.

(4) 710MMP1-siRNA-pAcGFP1-N3 Vector

5′ end of SEQ ID NO. 7 in Table 1 was dephosphorylated and then bound to HindIII cut site (in italics) to form a forward primer (5′-AGCTGCTACACCTTCAGTGGTGATGTTCA-3′); 5′ end of SEQ ID NO. 8 was also dephosphorylated and then bound to BamHI cut site (in italics) to form a reverse primer (5′-GATCMAACATCACCACTGAAGGTGTAGC-3′). 5 μl of the forward primer and 5 μl of the reverse primer were annealing at 75° C. in PCR to form a double strand. 4 μl double strand and 2 μl pAcGFP1-N3 were ligated at 4° C. to obtain a 710MMP1-siRNA-pAcGFP1-N3 vector.

(5) 742MMP1-siRNA-pAcGFP1-N3 Vector

5′ end of SEQ ID NO. 9 in Table 1 was dephosphorylated and then bound to HindIII cut site (in italics) to form a forward primer (5′-AGCTCAGGATGACATTGATGGCATCCAAGG-3′); 5′ end of SEQ ID NO. 10 was also dephosphorylated and then bound to BamHI cut site (in italics) to form a reverse primer (5′-GATCCCTTGGATGCCATCAATGTCATCCTG-3′). 5 μl of the forward primer and 5 μl of the reverse primer were annealing at 75° C. in PCR to form a double strand. 4 μl double strand and 2 μl pAcGFP1-N3 were ligated at 4° C. to obtain a 742MMP1-siRNA-pAcGFP1-N3 vector.

(6) Analysis and Sequencing of Vectors

E. coli Top 10 F′ (Invitrogen) was transformed with each of 357MMP1-siRNA-pAcGFP1-N3 vector, 710MMP1-siRNA-pAcGFP1-N3 vector, and 742MMP1-siRNA-pAcGFP1-N3 vector and selected with kanamycin for extracting and nucleic acid sequencing.

### Preparation Example 3

### Selection of siRNA by Green Fluorescent Protein (GFP) Report System

MeWo cells obtained from Food Industry Research and Development Institute (Taiwan) (BCRC 60540) were transfected with each of 357MMP1-siRNA-pAcGFP1-N3 vector, 710MMP1-siRNA-pAcGFP1-N3 vector, and 742MMP1-siRNA-pAcGFP1-N3 vector separately. Whereas green fluorescent protein (GFP) gene under target gene downstream, the MeWo cells transfected with the above-mentioned vectors would express green fluorescent due to active target gene. Alternatively, siNA can directly silence MMP1 transcript resulting in no GFP expression.

About 1×10⁶ of MeWo cells were seeded in 24-wells plates under 37° C., 5% CO₂ for 24 hours prior to vector transfection. For transfection, the cells were treated with 25 μl Xfect™ Transfection Reagent (Clontech Laboratories) and plasmid DNA through 24 hours, and then MMP1 siRNA was also co-transfected into MeWo cells. After 48 hours, cells were monitored for GFP level, for example, a fluorescence microscopy (Olympus CKX41), wherein the excitation was 475 nm and emission was 505 nm. The control group is negative control siRNA with vector co-transfection.

### Preparation Example 4

### Quantitative PCR (Real-Time PCR)

Accordingly, MeWo cells were transfected directly with the above-said silence MMP1 siRNA, which were selected by the above GFP report system. After 24 hours, the cells were collected for reverse transcription by iScrip™ cDNA Synthesis Kit (Bio-rad) to obtain a cDNA, wherein the forward primer and the reverse primer were as shown in SEQ ID NO. 11, SEQ ID NO. 12. The cDNA was quantitated by iQ™ SYBR® Green Supermix (Bio-rad). The relative expression of MMP1 mRNA was divided by glyceraldehyde 3-phosphate dehydrogenase (GAPDH) and compared with control group by relative quantification (ddCT study) in statistics.

### Preparation Example 5

### Western Blotting

Accordingly, MeWo cells transfected with the above-said silence MMP1 siRNA were collected for total protein after 48 hours to observe MMP1 inhibition by image analysis system (Fusion-SL2-3500WL).

### Example 1

### Selection siRNA by Report System

The MeWo cells were treated with Xfect™ Transfection Reagent and MMP1-pAcGFP1-N3 vector for 24 hours, 48 hours or 72 hours to observe fluorescence changes. The fluorescent expression of cells was increasing in coordination with the increasing incubation time. However, the fluorescent expression was not ideal due to that the inserted gene was too long to affect protein folding. Therefore, in order to shorten the length of target gene, target gene 357MMP1, 710MMP1, and 742MMP1 were constructed separately to pAcGFP1-N3 plasmid, wherein target genes 357MMP1, 710MMP1, and 742MMP1 were presented as siRNA complementary to specific sequence. Since the length of target gene was only 30 bp, forward primer and reverse primer were annealed by PCR machine to form a double strand and ligated to pAcGFP1-N3 vector cut by HindIII and BamHI.

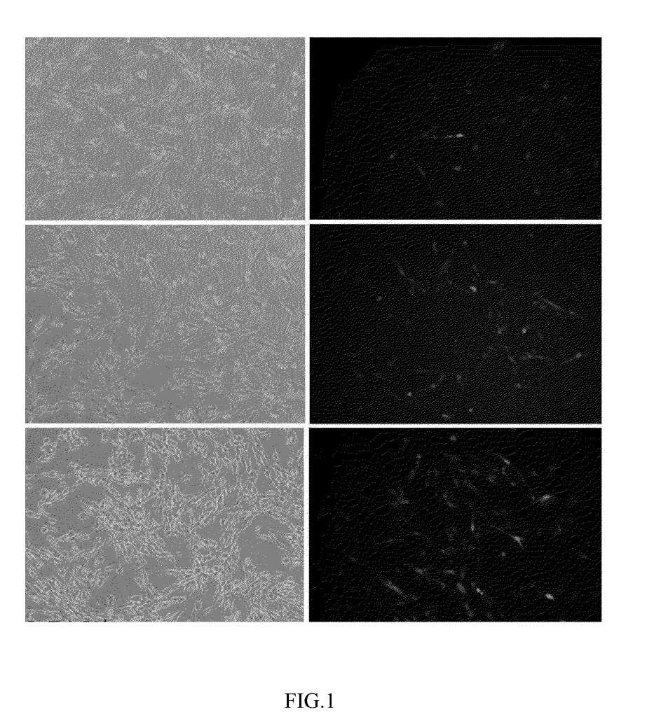

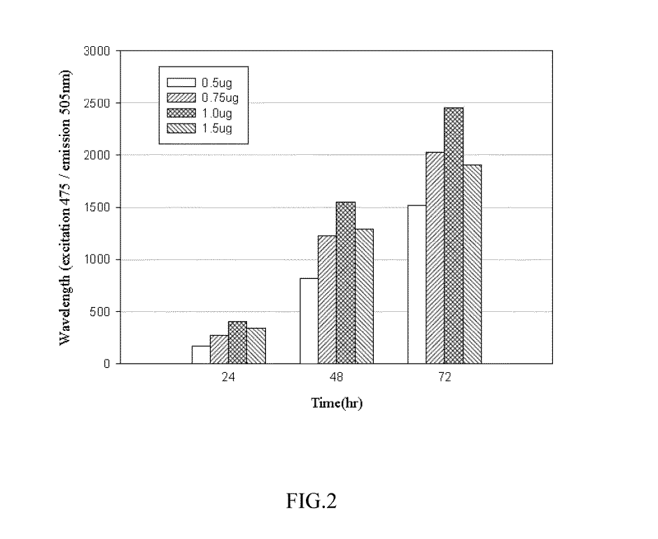

As shown in FIG. 1, MeWo cells were treated with 25 μl Xfect™ Transfection Reagent and 357MMP1-pAcGFP1-N3 vector for 24 hours, 48 hours, and 72 hours, and the fluorescent expression was increasing in coordination with the increasing incubation time. As shown in FIG. 2, the fluorescent expression was increasing in coordination with the increasing vector concentration (0.5 μg, 0.75 μg, 1.0 μg, and 1.5 μg). Accordingly, the best concentration of vector was 1.0 μg, and the highest fluorescent expression can be observed at 72 hours incubation time.

357MMP1-siRNA-pAcGFP1-N3 vector, 710MMP1-siRNA-pAcGFP 1-N3 vector, and 742MMP1-siRNA-pAcGFP1-N3 vector obtained from preparation example 2, and target siRNA (representing 357siRNA, 710siRNA, 742siRNA) or control group siRNA (representing negative siRNA) (Invitrogen), wherein a content of GC of the control Group siRNA was 48%, which was similar to a content of GC of target siRNA between 45% and 55%, were transfected separately into MeWo cells. Since Xfect™ Transfection Reagent would cause cells toxicity and affect fluorescent expression, cell viability was examined by MTT reagent after the treatment of fluorescent assay to exclude deviation. The fluorescent expression of each cell was obtained from the fluorescent expression divided by cell viability, and the fluorescent expression of control group can be used as background to ensure non-specific complementation or other genes inhibition.

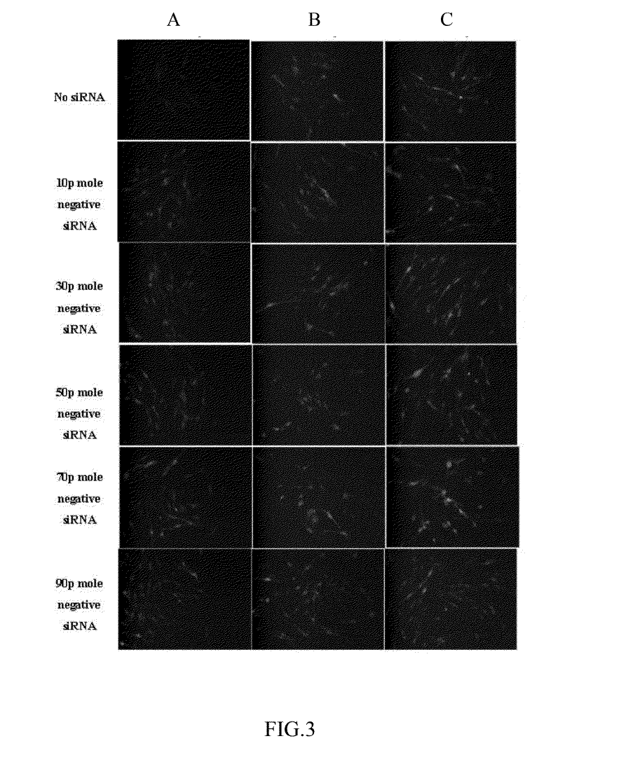

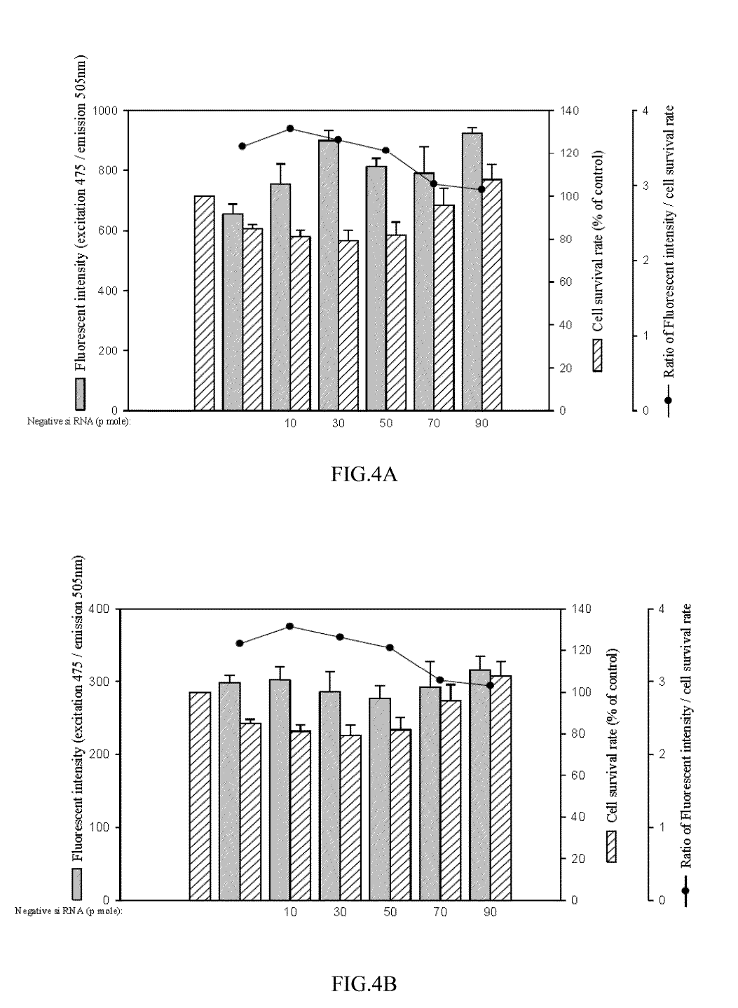

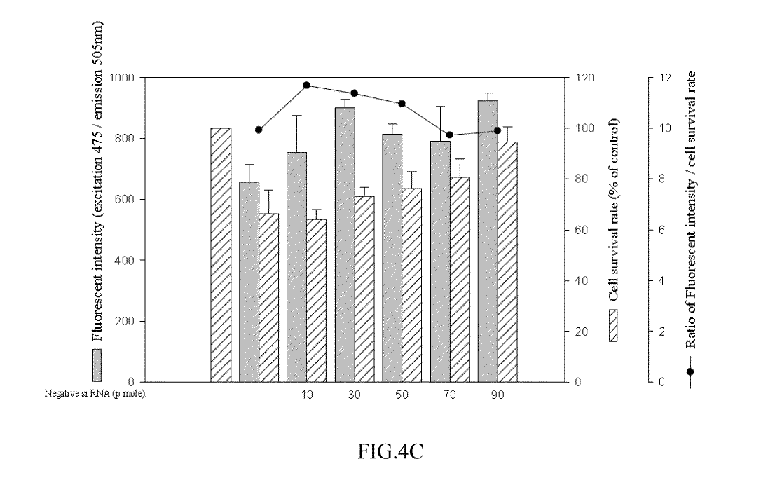

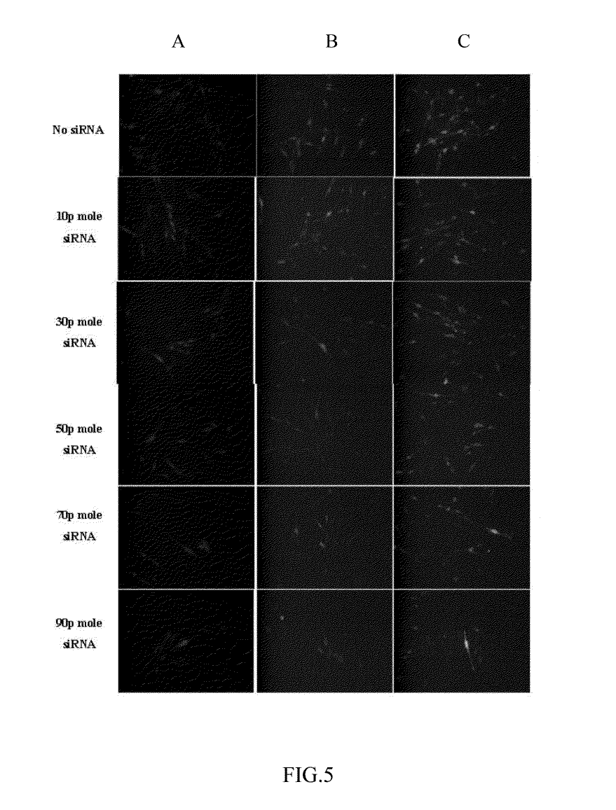

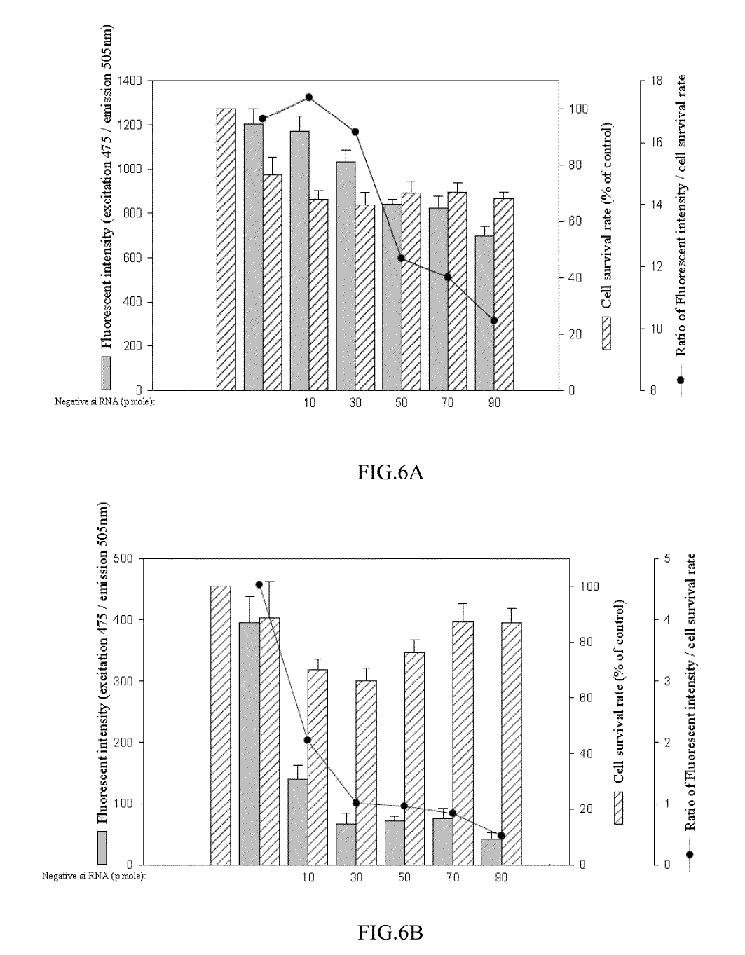

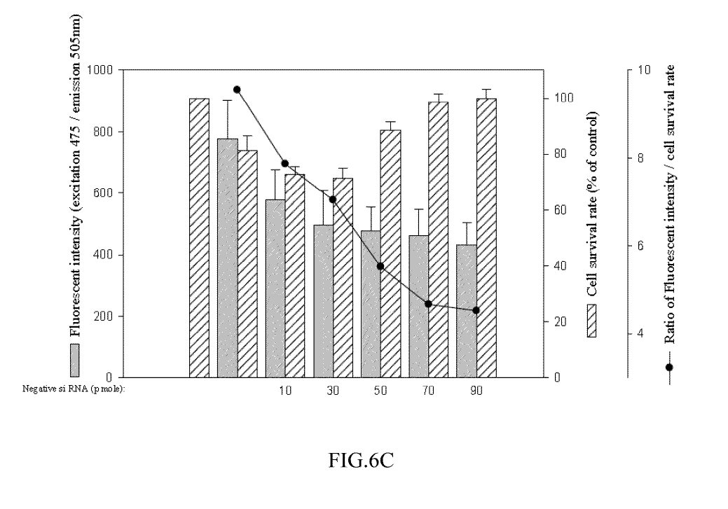

As shown in FIG. 3 to FIG. 4C, while MeWo cells were transfected with control siRNA, the fluorescent intensity and cell survival rate had no significant change. As shown in FIG. 5 to FIG. 6C, the fluorescent expression was decreasing in coordination with the increasing siRNA concentration. It means that three target siRNAs were capable of specific and distinct gene silencing, wherein the inhibition of 357siRNA was 39.2%; the inhibition of 710siRNA was 89.4%; the inhibition of 740siRNA was 54.1. Accordingly, the best interference effect was 710siRNA, and the inhibition was about 90%.

### Example 2

### Interference or Inhibition Endogenous MMP1 Gene Expression by Gene Silencing siRNA

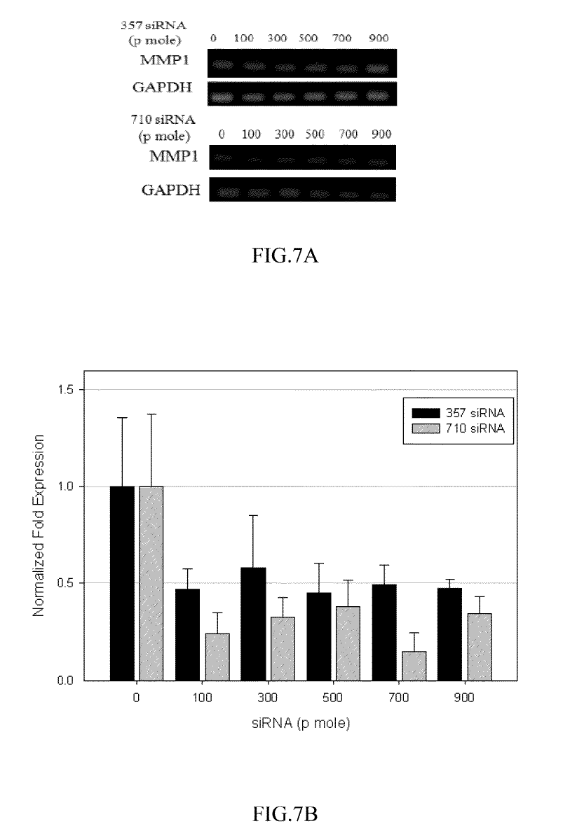

Quantitative PCR (real-time PCR) was prepared for analysis from preparation example 2. As shown in FIG. 7A and FIG. 7B, the MeWo cells were transfected with 357siRNA or 710siRNA of various concentrations (100 pmole, 300 pmole, 500 pmole, 700 pmole or 900 pmole) separately, and then the DNA of MeWo cells were analyzed by real-time quantitative polymerase chain reaction (RT-PCR) and agarose gel electrophoresis analysis. Accordingly, endogenous MMP1 gene can be inhibited by 357siRNA and 710siRNA, but siRNAs of various concentrations were not dose-dependent in accordance with inhibition, which might be attributed to siRNA half life and instability. The endogenous MMP1 gene inhibition of 357siRNA and 710siRNA were 55% and 85%. Thus, 710siRNA had better inhibition efficiency, and the result corresponded to example 1.

### Example 3

### Interference or Inhibition Endogenous MMP1 Protein Expression by Gene Silencing siRNA

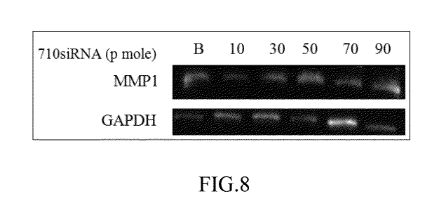

As shown in FIGS. 7A to 7B, the dose-dependence would be affected when the concentration of siRNA was higher than 100 pmole. Western blotting was prepared for MMP1 protein inhibition analysis from preparation example 5. As shown in FIG. 8, the MeWo cells were transfected with 710siRNA of various concentrations (10 pmole, 30 pmole, 50 pmole, 70 pmole or 90 pmole) separately. Accordingly, the endogenous MMP1 protein inhibition of 90 pmole 710siRNA was 89.4%.

What is claimed is:

1. An interference ribonucleic acid for inhibiting MMP1, which is composed of 19 to 25 nucleotides, and has a sequence essentially identical to the sequence as set forth in SEQ ID NO: 5 or the sequence as set forth in SEQ ID NO: 6.

2. An interference ribonucleic acid for inhibiting MMP1, which is composed of 19 to 25 nucleotides, and has a sequence essentially identical to the sequence as set forth in SEQ ID NO: 7 or the sequence as set forth in SEQ ID NO: 8.

3. An interference ribonucleic acid for inhibiting MMP1, which is composed of 19 to 25 nucleotides, and has a sequence essentially identical to the sequence as set forth in SEQ ID NO: 9 or the sequence as set forth in SEQ ID NO: 10.

4. A method of inhibiting MMP1 in a subject in need thereof, comprising: administering an effective amount of an interference ribonucleic acid having a length of 19 to 25 nucleotides to the subject, wherein the interference ribonucleic acid has a sequence selected from the group consisted of the sequences as set forth in, or essentially identical to, SEQ ID NO: 5, SEQ ID NO: 6, SEQ ID NO: 7, SEQ ID NO: 8, SEQ ID NO: 9, and SEQ ID NO: 10.

5. A pharmaceutical composition for anti-cancer metastasis comprising an effective amount of interference ribonucleic acid, and a pharmaceutically acceptable carrier, wherein the interference ribonucleic acid has a sequence selected from the group consisted of the sequences as set forth in, or essentially identical to, SEQ ID NO: 5, SEQ ID NO: 6, SEQ ID NO: 7, SEQ ID NO: 8, SEQ ID NO: 9, and SEQ ID NO: 10.

6. The pharmaceutical composition as claimed in claim 5, wherein the effective amount of the interference ribonucleic acid is 0.5 μg to 1.5 μg.

7. The pharmaceutical composition as claimed in claim 5, wherein the cancer is lung cancer, prostate cancer, breast cancer, colon cancer, gastric cancer, pancreatic cancer, liver cancer, esophageal cancer, brain tumor, ovarian cancer, uterine cancer, melanoma, kidney cancer, head and neck tumor, skin cancer, bladder cancer, osteosarcoma, biliary tract cancer, vulvar cancer, testicle tumor, penis cancer, rectal cancer, mediastinal tumor, urethra cancer, villous cancer, thyroid cancer, parathyroid carcinoma, pheochromocytoma, blastocytoma, malignant lymphoma, leukemia or multiple myeloma.

8. The pharmaceutical composition as claimed in claim 7, wherein the skin cancer is basal cell carcinoma (BCC), squamous cell carcinomas (SCC) or melanoma.


SEQUENCE LISTING (12 sequences)

SEQ ID NO: 1 - DNA, 23 bp, artificial
  synthesized
atgcacagctttcctccactgct

SEQ ID NO: 2 - DNA, 29 bp, artificial
  synthesized
tcaatttttcctgcagttgaaccagctat

SEQ ID NO: 3 - DNA, 42 bp, artificial
  synthesized
aagcttgccgccaccatgggtagctttcctccactgctgctg

SEQ ID NO: 4 - DNA, 45 bp, artificial
  synthesized
ggatccgatgggctggacaggattttgggaacgtccatatatggc

SEQ ID NO: 5 - DNA, 30 bp, artificial
  synthesized
agctcacgccagatttgccaagagcagatc

SEQ ID NO: 6 - DNA, 30 bp, artificial
  synthesized
gatcgatctgctcttggcaaatctggcgtg

SEQ ID NO: 7 - DNA, 29 bp, artificial
  synthesized
agctgctacaccttcagtggtgatgttca

SEQ ID NO: 8 - DNA, 29 bp, artificial
  synthesized
gatctgaacatcaccactgaaggtgtagc

SEQ ID NO: 9 - DNA, 30 bp, artificial
  synthesized
agctcaggatgacattgatggcatccaagg

SEQ ID NO: 10 - DNA, 30 bp, artificial
  synthesized
gatcccttggatgccatcaatgtcatcctg

SEQ ID NO: 11 - DNA, 22 bp, artificial
  synthesized
agtcaagtttgtggcttatgga

SEQ ID NO: 12 - DNA, 20 bp, artificial
  synthesized
ttgtcactgaagctgctctc